(19) United States
(12) Patent Application Publication
(10) Pub. No.: US 2018/0035832 A1
(43) Pub. Date: Feb. 8, 2018

(54) Blanket

(71) Applicant: Tina Ureten, Toronto (CA)

(72) Inventor: Tina Ureten, Toronto (CA)

(21) Appl. No.: 15/417,308

(22) Filed: Jan. 27, 2017

(30) Foreign Application Priority Data
Aug. 2, 2016 (CA) 2937789

(51) Int. Cl. A47G 9/02; A61M 21/02

(52) U.S. Cl. CPC A47G 9/0223; A61M 21/02; A47G 2009/004

(57) ABSTRACT

A blanket sleep aid is disclosed consisting of two fabric sheets quilted together by a pattern of stitching to form a quilted blanket having plurality of closed pockets extending contiguously from the sides and ends of the blanket. The pockets enclosing a quantity of beads with a total cumulative total weight of between 10 to 12 percent of the body weight of the user. The beads are equally dispersed among the pockets such that each pocket has an equal weight of beads. An envelope formed on a first end of the blanket, the envelope having a an internal space, an opening for accessing the internal space, a flap for closing off the opening and a closure for holding the flap in a closed position, and aromatherapy satchel configured to fit within the envelope, the aromatherapy satchel consisting of a porous flexible sheet imbued with aromatherapy oil.

### FIELD OF THE INVENTION

The invention relates generally to sleeping blankets.

### BACKGROUND OF THE INVENTION

Despite the advances in modern medicine and improved living standards, there are some very common health problems which has enormous impact on the productivity and well being of the whole society. Many of the problems relating to physical and psychological health relate directly to a lack of sleep. It has been well documented that a lack of sleep can have profoundly negative consequences for the health, productivity and emotional well being of individuals. As a result, various sleep aids, both pharmaceutical and non-pharmaceutical, have been used to provide individuals with improved sleep. Unfortunately, there are dangers with the chronic use of pharmaceutical sleep aids, and most non-pharmaceutical sleep aid methods are not very effective. An improved non-pharmaceutical sleep aid method is therefore required.

### SUMMARY OF THE INVENTION

In accordance with one aspect of the present invention, there is provided a blanket for use in helping a person fall asleep. The blanket includes first and second fabric sheets each having equal widths and lengths, the first and second fabric sheets being quilted together by a diamond pattern of bindings to form a quilted blanket having plurality of closed pockets between the first and second fabric sheets. Each of the closed pockets enclose a space containing a quantity of beads, the quantity of beads being selected from the group consisting of tourmaline ceramic beads, alkaline ceramic beads, maifan stone ceramic beads, far infrared ceramic beads, and negative ion ceramic beads. One of the first and second fabric sheets being made of a bamboo charcoal nanotechnology fabric. An envelope formed on the blanket, the envelope having a an internal space, an opening for accessing the internal space, a flap for closing off the opening and a closure for holding the flap in a closed position. Finally, an aromatherapy satchel is profiled which is configured to fit within the envelope, the aromatherapy satchel consisting of a porous flexible sheet imbued with aromatherapy oil.

In accordance with another aspect of the present invention, there is provided a blanket for use in helping a person fall asleep. The blanket includes first and second fabric sheets each having equal widths and lengths, the first and second fabric sheets being quilted together by a diamond pattern of binding to form a quilted blanket having plurality of closed pockets formed between the first and second fabric sheets. Each of the closed pockets encloses a space containing a quantity of beads. The quilted blanket also includes an envelope formed on a first end of the blanket, the envelope having a an internal space, an opening for accessing the internal space, a flap for closing off the opening and a closure for holding the flap in a closed position. Finally, the blanket further includes an aromatherapy satchel configured to fit within the envelope, the aromatherapy satchel consisting of a porous flexible sheet imbued with aromatherapy oil.

In accordance with another aspect of the present invention, there is provided a blanket for use in helping a person fall asleep. The blanket includes first and second fabric sheets each having equal widths and lengths, the first and second fabric sheets being quilted together by a pattern of bindings to form a quilted blanket having plurality of closed pockets formed between the first and second fabric sheets. The quilted blanket has opposite first and second ends and opposite first and second sides, the pattern of binding configured such that the pockets extend contiguously from the first and second ends and between the first and second sides. Each of the closed pockets encloses a space containing a quantity of beads, the quantity of beads in all of the pockets having a cumulative total weight of at least 10% of a body weight of the person intending to sleep with the blanket. The beads are equally dispersed among the pockets such that each pocket has an equal weight of beads. The blanket also includes an envelope formed on a first end of the blanket, the envelope having a an internal space, an opening for accessing the internal space, a flap for closing off the opening and a closure for holding the flap in a closed position. Finally, an aromatherapy satchel is provided which is configured to fit within the envelope, the aromatherapy satchel consisting of a porous flexible sheet imbued with aromatherapy oil.

With the foregoing in view, and other advantages as will become apparent to those skilled in the art to which this invention relates as this specification proceeds, the invention is herein described by reference to the accompanying drawings forming a part hereof, which includes a description of the preferred typical embodiment of the principles of the present invention.

### DESCRIPTION OF THE DRAWINGS

FIG. 1 is a top view of a blanket made in accordance with the present invention.

FIG. 2 is a cross sectional view taken along line A-A in FIG. 1.

In the drawings like characters of reference indicate corresponding parts in the different figures.

### DETAILED DESCRIPTION OF THE INVENTION

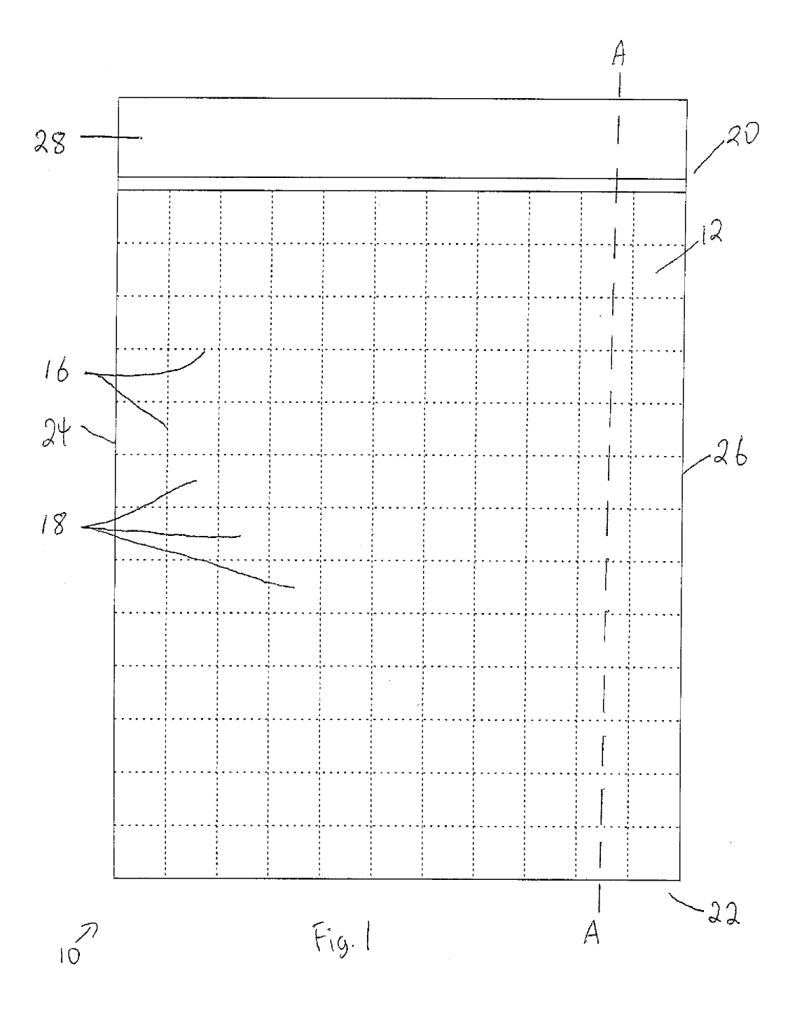

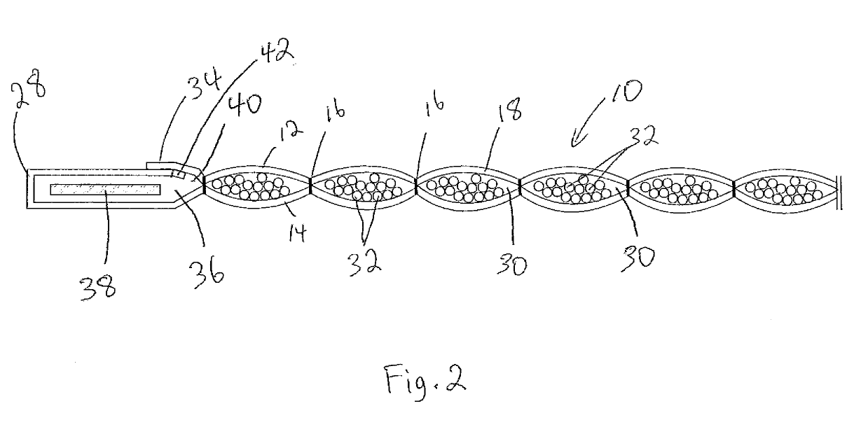

Referring to FIG. 1 a sleeping aid blanket made in accordance with the present invention, shown generally as item 10, consists of a roughly rectangular blanket made of an upper sheet 12 and a lower sheet 14 (see FIG. 2) which are stitched together by a pattern of bindings 16 to form a plurality of enclosed pockets 18 formed between the upper and lower sheets. Blanket 10 has first end 20 and opposite second end 22 and opposite sides 24 and 26. An elongated envelope 28 is formed at end 20.

The pattern of stitches is preferably configured such that pockets 18 form a contiguous quilt from side 24 to 26 and from end 20 to end 22. The pattern of bindings 16 preferably consists of stitches; however, other methods of binding may be used such as gluing, bonding or fabric welding. A diamond pattern or grid pattern of binding is preferably used, so that the pockets are formed contiguously, i.e. with adjacent pockets in contact with each other. It is possible to come out with other pattern bindings, such as a hexagonal pattern, which are capable of producing a contiguous pattern of pockets or approximately equal dimensions.

Referring now to FIG. 2, blanket 10 consists of a series of contiguous pockets 18 separated by bindings (stitches) 16. The pockets are relatively small compared to the blanket, preferably in the range of 2″×2″ in dimension. Each pocket 18 encloses a space 30 which is filled with a plurality of beads 32. Preferably the total cumulative total weight of all of the beads in the blanket adds up to at least 10% of the body weight of the person intending to use the blanket. Since the average person in the United States and Canada weights between 150 lbs to 200 lbs, the total cumulative weight of the beads in the bed will be at least in the range of 15 to 20 lbs.

Elongated envelope 28 has an opening 40 which is covered over by a flap 34 which can close over opening 40 and held in place by closure 42. Closure 42 preferably consists of a strip of hook and loop fastener such as Velcro™, but it may also consist of an elongated zipper or a row of buttons or some other closure which is configured to permit flat 34 to be held in place when closed but which can be opened to access interior 36 of envelope 28. Contained within interior 36 is an elongated satchel 38 which is impregnated with aromatherapy oils. Preferably the fabric forming envelope 28 is sufficiently porous that scent emanating from satchel 38 can pass through envelope 28. Satchel 38 may consist of a fabric or paper sheet which has been impregnated with aromatherapy oils. Several different aromatherapy oils are commercially available. By placing satchel 38 in envelope 28 positioned at one end of the blanket, the blanket can be worn over the user with the envelope close to the user's head, thereby releasing aromatherapy vapors/scent close to the user's nose. This permits lower quantities of aromatherapy oils to be used.

The therapeutic benefit of using aromatherapy (botanical) extracts has been recognized by mankind going as far back as cultures such as the Aborigines of Australia, the Native American Indians, and to the tribes in Africa. In more modern history, companies have produced commercial lines of extracted essential oils for topical and aroma therapeutic use. Essential Oils (Aromatherapy Oils) are the highly concentrated, volatile, aromatic essences of plants. Essential Oils are extracted directly from a variety of flowers, fruits, leaves, barks, roots, herbs, and spices. Most common essential oils known for their therapeutic sleep benefit are lavender, chamomile, sage, ylang ylang, bergamot, marjoram and cedarwood.

One study that mixed lavender, roman chamomile, and neroli in a 6:2:0.5 ratio discovered that the inhalation of this blend was more effective than traditional nursing methods at reducing the stress and improving the sleep of patients in an intensive care unit. Another study mixed lavender, ylang-ylang, marjoram, and neroli and concluded that it could lower blood pressure and improve sleep of middle aged women with hypertension. A further study reported that a blend of lavender, basil, juniper, and marjoram increased the likelihood that elderly hospitalized patients would experience sleep satisfaction by 20%. (Source: http://www.healthyandnaturalworld.com/the-best-essential-oils-for-insomnia/#more-12133).

It had been discovered that weighted blankets can help subjects sleep. Some trials from Sweden and Australia have proven that blankets with a weight of at least 10 percent of body weight can provide better sleep and improve many health conditions. A study conducted in 2014 found that, among 63 children ages 5 to 16 who slept with a weighted blanket and a non-weighted blanket, the overall preference was the weighted blanket. Additionally, two studies published in Occupational Therapy in 2008, reported that weighted blankets eased anxiety. For adults, a good weighted blanket is 15-30 pounds or 10-12% of the total body weight.

The weight in the blanket brings pressure to the touch points and acts like a massager. The stimulated touch points send the message to brain to release serotonin hormone. Serotonin is known for its relaxation effect on the body. It is the body's “feel good” substance. It contributes to well being and happiness and can regulate mood, mental health, memory and sleep.

The present invention significantly improves on prior attempts at producing weighted blankets by providing a design which distributes the weight of the blanket uniformly across the user's body. As mentioned above, pockets 18 are relatively small (preferably about 2″×2″) and are each filled with a quantity of beads so as to add weight to the blanket. The small pocket size effectively distributes the weight of the beads evenly across the surface of the blanket, creating a more uniform weight applied to the body when the blanket is draped over or wrapped around the sleeper. This uniform application of weight across the sleeper's body has been discovered to significantly aid in the user reaching sleep. The weight is created by using ceramic beads which are used to loosely fill the compartments in the blanket. The compartments are loose enough to let the beads move around with the body movements. That would make the beads to gather at one spot and increase the weight per unit tissue. This is an important fact to stimulate the touch points in the body while sleeping under the blanket. The stimulation of different points would send the necessary signals to body in order to secrete serotonin.

The weight of the blanket in this invention comes from ceramic beads, which haven't been used for the weight blankets used for the trials yet. The beads would be evenly distributed into the sewn compartments (pockets 18) and provide health benefits resulting from the emission of far infrared radiation (FIR) during sleep time. It is very well known the visible light spectrum from the sun is good for bones, increases the well being and improves the health. Similarly invisible wavelength of the sunlight like the far-infrared ray also provides many benefits to living organisms. FIR is a form of natural electromagnetic waves in the wavelength range of 4 to 1000 microns. A narrow spectrum between 4-14 microns FIR waves are particularly beneficial and easily absorbed by the body. Some natural minerals and human body can also generate FIR waves. It is shown that exposure to FIR promote circulation, increases tissue oxygen levels, enhances cell vitality and regulates body's performance. Histological findings revealed greater collagen regeneration and infiltration of fibroblasts that expressed transforming growth factor-p 1 (TGF-β1) in wounds with FIR irradiation. Subsequent studies have proved its vasodilator effect, which means it promotes circulation therefore improves the function of the organisms. Especially Chinese Medicine commonly uses infrared light as therapeutic treatment for conditions ranging from congestive heart failure to muscle tears and rheumatoid arthritis. The following effects of FIR emitting materials on human body are reported.
Increased blood flow
Faster wound healing
Regulation of the nervous system,
Decreased fatigue, increase energy
Anti-oxidant, anti-bacterial effect
Enhanced cell metabolism
Decreased cancer cell growth
Increased Collagen production
Relieving inflammation on joints and muscles
Increased viability of osteoblastic cells (slowing the process of Rheumatoid arthritis and osteoporosis)
Promote sleep quality
Improved skin health,
Slowed aging process

The beads used to fill pockets 18 are preferably selected for the ability to emit far infrared radiation. Tourmaline, maifan stone, silicone, germanium, tourmaline, jade and maifan stone are natural elements which naturally generate FIR radiation. They emit FIR even when they are in fine powder form. The powder is mixed with the ceramic clay to produce different size beads. With being close to the skin the infra red energy coming from the blanket filled with beads will boost the blood flow to the tissues. By increasing oxygen level FIR stimulates cell performance and regeneration, increases muscle strength and healing. Therefore FIR releasing beads relieve the pain, promote the sleep, regulate cellular functions. They also, absorb moisture to maintain dryness and eliminate odor by high absorption capacity.

The size of the beads are preferably 3-5 mm and their weight are heavy enough to alter tactile inputs of the body and increase serotonin level in the brain which has relaxation and anti-depressive effects. Different kinds of beads are utilized to fill the blanket and create weight. Each kind of bead brings different health benefits during the contact with the body and emitting FIR. The blending of different beads can be customized by increasing or decreasing the FIR effect to meet the needs of specific situations. The following is a list of the preferred types of beads which can be used, either alone or in combination, to make the blanket of the present invention.

Tourmaline Ceramic Beads:

Tourmaline Ceramic Balls produce more tourmaline effects than pure tourmaline due to the high absorbency of ceramic. Tourmaline ceramic balls advantages:
Produces Negative Ions, which are also known as air vitamins. They can help to regulate the body's ion balance therefore improve cellular functions, motivate natural healing. Negative ions also control the oxidation of the body. In today's environment, many factors can produce positive ions, which cause stress for the body, making negative ions more essential.
They also radiate far infrared which can penetrate to deeper parts of the body and promote blood circulation and regulates the metabolism. Tourmaline has a variety of natural minerals, many of which are very necessary minerals for cellular function.

Far Infrared Ceramic Beads

Far infrared ceramic beads are a type of commercially available ceramic bead which contain more than 10 different kinds of minerals which emit far infrared rays and activate the living organism's metabolism. They also diminish the heavy metals in the environment. They contain zinc, lithium, iodine, selenium and over 20 other trace elements. They have been promoted as being able to regulate cell metabolism and improve the body's immune capacity.

Alkaline Ceramic Beads

Alkaline beads are a commercially available beads made of natural stones through the nanometer processing which can continuously produce negative ions. Their main minerals are aluminium and zirconia with the forms of Al2O3, SiO2, CaO, MgO, Fe2O3 and others. They are widely used in the food industry and for water treatment.
When added into the water these beads increase PH, make alkaline water which is better for balancing body's PH. They emit high capacity of calcium therefore enhance the bone regeneration. They also eliminate inorganic minerals.

Maifan Stone Ceramic Beads

These beads consist of K, Ca, Mg, Si, Fe, Zn, Cu, Mo, Se, Mn, Sr, Ni, V, Li, Co, Cr, I, Ge, and Ti. Maifan Stone is a kind of non metal mineral, which is widely used for its strong adsorption ability to absorb heavy metal ions and toxins. It can remove mercury, lead, cadmium, arsenic and other heavy metals from the environment. It can also inhibit the reproduction of bacteria.

Quartz Beads

Clear Quartz is also known as Crystal Quartz or Rock Crystal is a natural crystal composed of silicon dioxide. Clear quartz is the most common, but white or rose quartz forms when it is been in places where frost can exist. Quartz atoms actually form in perfect double-helix patterns like in our DNA. Quartz has been used for sacred power object by many cultures, including Native Americans, Tibetan monks, and priests of Christian Europe. Some other cultures have been using quartz for healing, meditations and spiritual development since the ancient times. It is believed that quartz can enhance person's abilities to enjoy life and amplify concentration, reduce stress, help with balancing or calming by strengthening inner healing capacities.

Quartz is one of the most common mineral on earth, which resonates with the frequencies of the silica in our body. This helps to energize, harmonize and balance the energies of the body and to promote and restore well being. Clear Quartz has both pyroelectric and piezoelectric properties. Pyroelectricity is its ability to generate an electrical charge when heated or cooled, with these qualities quartz crystal has been used in making marine instruments, clocks, watches, and the radio. While the piezoelectric properties of clear quartz crystal enables it to develop an electrical charge result from the application of mechanical stress. Clear quartz acts as a receiver and transmitter, it absorbs, stores, releases and regulates the energies. When crystals absorb the energy, it dissipates by different vibrations of all the crystals vibrating together act as a natural form of cleansing.

Quartz crystals don't have immediate effect on the user's body, it needs hours of contact until it becomes akin to person's frequency of energy. Using quartz crystal pieces as filler in weight blanket serves to obtain the best benefit from its healing features during the sleep.

Negative Ion Ceramic Beads

Negative ion ceramic beads are made from natural stones through the nanometer processing which can be continuously produced negative ions. They can release negative ions at a rate of 1000/CM³˜20000/CM³ depending on different composition. They can be used to kill bacteria in the water, increase dissolved oxygen, and activate water. They increase negative ion concentration in the air and used for improving air quality. They prevent “Air conditioning disease” and the “computer syndrome. Negative ion ceramic beads can also be used in the car, to eliminate the any smell and clean the air. They discharge high capacity of negative ions and emit FIR. Their effect on the body increases natural healing ability & increase immunity.

Sheets 12 and 14 are preferably made from a flexible yet strong fabric. Any flexible fabric can be used to form sheets 12 and 14, including cotton, wool or even synthetic fabrics; however, bamboo charcoal nanotechnology fabric (BCNF) is particularly well suited for making the sheets. BCNF is commercially available from several different suppliers. The bamboo charcoal component in the fabric is created by heating bamboo at temperatures of 800 degrees and then the charcoal itself is processed and mixed in with fibers. The textile's softness, durability, water resistance and breathability can be enhanced with the production method. Bamboo charcoal pieces can be added to the spinning solution to be incorporated into the fibers, and then spun is turned into yarn and woven to form this special fabric. BCNF has many benefits for the user. It emits far infrared radiation (FIR), increases the blood circulation of the user, absorbs odor and moisture so as to keep the body dry. It also eliminates unwanted electromagnetic field created around electronic devices; therefore, if the blanket is kept between the laptop/tablet and the body the electromagnetic waves from the device won't be able to reach the body. This is a big advantage in using this sort of fabric as with today's lifestyle we spend quite a lot of time close to electromagnetic fields and the long term harm of these electromagnetic fields is not known yet. The bamboo charcoal fabric has several other advantages which are well suited for use in the present application, including:

Easier on sensitive skin—The fabric inhibits bacterial metabolism causing fewer allergic skin reactions than other fibers sterilized with antimicrobial agents
Reduces Static buildup—Bamboo charcoal fabric is made of conductive material so it keeps a balanced charge in fabric to help reduce static buildup.
Superior Absorption & Deodorizing Ability—This trait is due to the highly porous structure of the bamboo fabric. Bamboo charcoal fiber's carbon content is nearly 93%˜96%, and this property enables to absorb sulfur and nitrogen based compounds.
Far Infrared Radiation properties—Because bamboo charcoal fiber contains natural elements such as potassium, magnesium, calcium as well as fibers which have property of far-infrared radiation. The fabric disperses the FIR radiation to activate human cells, increase cellular energy, accelerate the speed of blood circulation, and promote and improve body metabolism.
Superior Washing and Durability—Bamboo charcoal fibers have quick-drying feature. Because the bamboo charcoal nano particles are embedded in the fabric rather than simply coated onto the surface, the fabric can be washed numerous times with no adverse effect on the charcoal qualities.
Moisture Regulation—Bamboo charcoal textile has a cross-section filled with various micro-gaps and micro-holes so compared to conventional fabrics, it has better moisture absorption and ventilation qualities.
Thermal Regulation—The highly porous nature of bamboo charcoal also lets it act as an insulator against the cold while also retaining heat. According to some test results, when a 500 W halogen light shone on the bamboo charcoal yarn for 10 minutes, the yarn emits far more infrared rays 87%˜92%, and warm up temperature of 10 degrees Celsius, which is much higher than wool's 5 degrees Celsius.
Promotes Better Health—The Bamboo Charcoal fiber contains potassium, calcium and other minerals which attract negative ions for the benefit of cellular health.
Antimicrobial Functions—Bamboo Charcoal fiber effectively decomposes the micro-organisms attached to its surface and in the air around it. The American Association for Testing and Materials ASTME2149-2001 fixation antibacterial activity of antibacterial agents dynamic testing method is adopted to study Staphylococcus aureus ATCC6538 (gram-positive bacteria). And by testing the one-sided knitted fabric bamboo charcoal filament bacteriostatic antibacterial function, the results show that the inhibiting rate is 65%, antimicrobial rate is 84%.

The FIR generating material used in the construction of the beads has an additional property which can be quite useful, namely the ability to dampen electromagnetic waves. It is therefore possible to use blanket 10 as a pad separating a user from an electronic device such as a laptop computer, a tablet computer or even a cell phone. In an alternate embodiment of the present invention, blanket 10 may be made smaller so that it can act as a smaller pad for placement on a user's lap and used as a “shield” to dampen electromagnetic waves emanating from any electronic equipment resting on the pad. If blanket 10 is to be formed as a smaller pad for covering only a portion of the body and shielding that portion of the body from electromagnetic waves, then the dimensions of pockets 18 may likewise be reduced from 2″×2″ to 1″×1″ or there about to ensure a more compact structure and improved flexibility.

Blanket 10 can also be used to aid the relief of sore muscles. Since the beads filling the blanket and the bamboo fabric forming the blanket are made of FIR generating materials, donning

The present invention has several advantages. The use of between 15 to 30 pounds of beads loosely packed into a plurality of small pockets formed contiguously across the blanket provides a sleep encouraging experience. The beads, being able to roll around in the pockets, and providing a large weight upon the body encourages the onset of sleep. The material of the sheets forming the blanket, and the nature of the beads themselves, has health promoting effects such as the generation of FIR. Finally, the aromatherapy satchel aids in the relaxation of the user helping the user to fall asleep more quickly. The cumulative and synergistic effect of these elements provides a blanket which can aid in the user's sleep.

A specific embodiment of the present invention has been disclosed; however, several variations of the disclosed embodiment could be envisioned as within the scope of this invention. It is to be understood that the present invention is not limited to the embodiments described above, but encompasses any and all embodiments within the scope of the following claims.

Therefore, what is claimed is:

1. A blanket for use in helping a person fall asleep, the blanket comprising;
a. A first and second fabric sheets each having equal widths and lengths, the first and second fabric sheets being quilted together by a diamond pattern of binding to form a quilted blanket having plurality of closed pockets between the first and second fabric sheets;
b. Each of said plurality of closed pockets enclosing a space containing a quantity of beads, the quantity of beads being selected from a group comprised of tourmaline ceramic beads, alkaline ceramic beads, maifan stone ceramic beads, far infrared ceramic beads, quartz beads, and negative ion ceramic beads;
c. One of the first and second fabric sheets being made of a bamboo charcoal nanotechnology fabric;
d. An envelope formed on the blanket, the envelope having a an internal space, an opening for accessing the internal space, a flap for closing off the opening and a closure for holding the flap in a closed position, and
e. An aromatherapy satchel configured to fit within the envelope, the aromatherapy satchel comprising a porous flexible sheet imbued with aromatherapy oil.

2. The blanket of claim 1 wherein the quilted blanket has opposite first and second ends and opposite first and second sides, the envelope formed as an elongated enveloped extending along the first end of the quilted blanket between the first and second sides.

3. The blanket of claim 2 wherein the opening extends along the first end of the quilted blanket and wherein the closure comprises a plurality of closure elements positioned along the flap.

4. The blanket of claim 1 wherein the beads have a cumulative weight equal to at least between 10% to 12% of a body weight of the person, the beads being equally distributed among the pockets such that each pocket has an equal weight of beads.

5. The blanket of claim 4 wherein the pockets form a contiguous quilt pattern from the second end to the envelope and from the first to second side of the quilted blanket.

6. A blanket for use in helping a person fall asleep, the blanket comprising;
a. A first and second fabric sheets each having equal widths and lengths, the first and second fabric sheets being quilted together by a diamond pattern of binding to form a quilted blanket having plurality of closed pockets formed between the first and second fabric sheets;
b. Each of said plurality of closed pockets enclosing a space containing a quantity of beads;
c. An envelope formed on a first end of the blanket, the envelope having a an internal space, an opening for accessing the internal space, a flap for closing off the opening and a closure for holding the flap in a closed position, and
d. An aromatherapy satchel configured to fit within the envelope, the aromatherapy satchel comprising a porous flexible sheet imbued with aromatherapy oil.

7. The blanket of claim 6 wherein the beads have a cumulative weight equal to at least between 10% to 12% of the person's weight, the beads being equally distributed among the pockets such that each pocket has an equal weight of beads.

8. The blanket of claim 7 wherein the pockets form a contiguous quilt pattern from the second end to the envelope and from the first to second side of the quilted blanket.

9. The blanket of claim 8 wherein the cumulative weight of the beads is between 15 lbs to 30 lbs.

10. The blanket of claim 9 wherein the beads are selected from the group of beads comprising tourmaline ceramic beads, alkaline ceramic beads, far infrared ceramic beads, maifan stone ceramic beads and negative ion ceramic beads.

11. The blanket of claim 9 wherein the cumulative weight of beads comprises a mixture of different beads selected from a group of beads comprising tourmaline ceramic beads, alkaline ceramic beads, far infrared ceramic beads, maifan stone ceramic beads, quartz beads, and negative ion ceramic beads.

12. A blanket for use in helping a person fall asleep, said person having a body weight, the blanket comprising;
a. A first and second fabric sheets each having equal widths and lengths, the first and second fabric sheets being quilted together by a pattern of stitching to form a quilted blanket having plurality of closed pockets formed between the first and second fabric sheets, the quilted blanket having opposite first and second ends and opposite first and second sides, the pattern of stitching configured such that the pockets extend contiguously from the first and second ends and between the first and second sides;
b. Each of said plurality of closed pockets enclosing a space containing a quantity of beads, the quantity of beads in all of the pockets having a cumulative total weight of at least between 10% to 12% of the body weight, the beads being equally dispersed among the pockets such that each pocket has an equal weight of beads;
c. An envelope formed on a first end of the blanket, the envelope having a an internal space, an opening for accessing the internal space, a flap for closing off the opening and a closure for holding the flap in a closed position, and
d. An aromatherapy satchel configured to fit within the envelope, the aromatherapy satchel comprising a porous flexible sheet imbued with aromatherapy oil.

13. The blanket of claim 12 wherein the cumulative weight of beads comprises a mixture of different beads selected from a group of beads comprising tourmaline ceramic beads, alkaline ceramic beads, far infrared ceramic beads, maifan stone ceramic beads, quartz beads and negative ion ceramic beads.

14. A flexible pad for covering over a part of a human body and partially shielding said part from electromagnetic radiation, the flexible pad comprising;
a. A first and second fabric sheets each having equal widths and lengths, the first and second fabric sheets being quilted together by a diamond pattern of binding to form a quilted blanket having plurality of closed pockets between the first and second fabric sheets;
b. Each of said plurality of closed pockets enclosing a space containing a quantity of beads, the quantity of beads being selected from a group comprised of tourmaline ceramic beads, alkaline ceramic beads, maifan stone ceramic beads, far infrared ceramic beads, quartz beads, and negative ion ceramic beads, and
c. One of the first and second fabric sheets being made of a bamboo charcoal nanotechnology fabric.
